(19) United States
(12) Patent Application Publication
(10) Pub. No.: US 2008/0071084 A1
(43) Pub. Date: Mar. 20, 2008

(54) Method for preparing alkyl ethers and aryl ethers

(76) Inventors: Andreas MEUDT, Hofheim (DE); Bernhard J. Rausch, Bad Soden (DE)

Correspondence Address: PROPAT, L.L.C., 425-C SOUTH SHARON AMITY ROAD, CHARLOTTE, NC, 28211-2841, US

(21) Appl. No.: 11/755,316

(22) Filed: May 30, 2007

(30) Foreign Application Priority Data
Jun. 7, 2006 (DE) 10 2006 026 431.2

(51) Int. Cl. C07D 333/32; C07D 239/02; C07D 241/02; C07D 307/02; C07D 333/34

(52) U.S. Cl. 544/239; 544/407; 544/308; 546/301; 548/543; 549/66; 549/475; 568/633; 568/662

(57) ABSTRACT

Method for preparing compounds of the formula (III) by reacting compounds of the formula (II) with a) an alcoholate or b) an alcohol R1-OH and a base

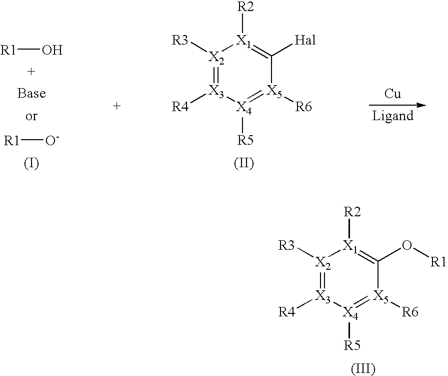

in the presence of a Cu-containing catalyst and of a ligand, where

X₁₋₅ are independently of one another either carbon or nitrogen, or in each case two adjacent X₁R₁, with i=1−6, linked by a formal double bond together O, S, NRH or _{Nrl}.

The ligands preferably employed are acyclic and/or cyclic oligo- and polyglycols, oligo- and polyamides or oligo- and polyamine glycols of the general formula (IV)

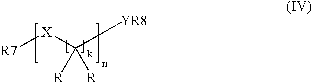

k is an integer >0 and n is an integer >1;

X and Y are independently of one another O, NH or NR₁.

### CROSS REFERENCE TO RELATED APPLICATIONS

This application claims priority to its parent application, German Patent Application 10 2006 026 431.2, filed Jun. 7, 2006, hereby incorporated by reference herein in its entirety.

### FIELD OF THE INVENTION

The invention relates to a method for preparing organic compounds by generating alcoholate compounds from alcohols and base or employing purchasable alcoholates and their reaction with suitable aryl halides in the presence of copper salts and suitable ligands (equation I).

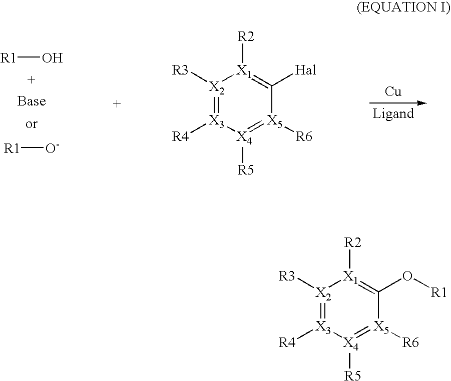

### BACKGROUND OF THE INVENTION

Copper-promoted coupling reactions have become of increasing importance in recent years during the course of the general upsurge in organometallic chemistry. Such synthetic methods are crucial especially in the preparation of compounds for the pharmaceutical and agrochemical industry, because it is possible thereby to assemble the increasingly complex structures of the required fine chemicals for the pharmaceuticals and agrochemicals sectors.

In this connection, the copper-promoted C,X-linkage reactions (X=heteratom), such as, for example, carbon—nitrogen couplings, carbon—oxygen couplings of carbon—sulfur couplings, provide a very versatile synthetic potential for assembling complex organic structures.

A large number of coupling reactions is known, with most linkage reactions being characterized by the following characteristics:
(1) Ligands which form a complex with the copper metal are added when carrying out the reaction. The ligands are in this case aromatic or aliphatic amides or alcohols.
(2) The reactions achieve conversions (determined by GC or HPLC) typically of from 30 to 80%, mostly between 50 and 70%. Considerable amounts of precursors (aryl halides) thus remain in the reaction mixture and must be removed by complicated workup methods.
(3) Typical reaction times are from 24 to 48 h, mostly between 36 and 42 h.
(4) After the synthesis has been carried out, the reaction mixture is typically subjected to an aqueous workup, in which case the organic ligands are often present together with the coupling product in the organic phase, frequently making an elaborate workup method such as, for example, column chromatography necessary.
(5) The reactions are carried out dilute in organic solvents, preferably using solvents such as 2-propanol, toluene, ethylene glycol, methanol, 1,4-dioxane, 1,2-dimethoxyethane, triethylamine or mixtures thereof.

One example of a copper-promoted C,O-linkage reaction is described by Buchwald et al., (WO 02/085 838 A1). In this approach, for example, 1-butoxy-3,5-dimethylbenzene is formed from 3,5-dimethyliodobenzene and n-butanol in the presence of CuI, Ca₂CO₃ and a ligand (equation 11). Typical ligands in this case are 2-phenylphenol, 2,6-dimethylphenol, 2-isopropylphenol, 1-naphthol, N,N-dimethylglycine, methyliminodiacetic acid and N,N,N′,N′-tetramethylenediamine. The yields achieved in this case are from 20 to 81% with a reaction time of 36 h at 105° C.

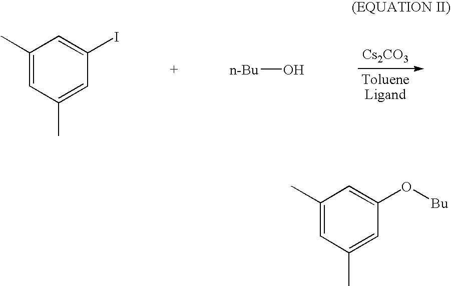

The best yields are achieved in this case with 2-phenylphenol (=2-hydroxybiphenyl), a compound which is classified as very environmentally toxic and as potentially carcinogenic (R40). This represents a considerable disadvantage of this method and makes it very difficult to employ this process—beyond the general problems associated with the industrial use of such CMR substances in manufacturing—for preparing pharmaceutical fine chemicals.

A further disadvantage of the method is that, after stopping the reaction by aqueous workup, the organic product phases are contaminated with the ligand. Ligands are frequently soluble in conventional organic solvents, meaning that they can be separated from the coupling products only by elaborate additional operations. In addition, owing to the high affinity of the metal for these ligands, the products often contain copper residues. This is likewise disadvantageous and uneconomic from the industrial manufacturing viewpoint.

In some cases, large amounts of solvent mixtures, some of which are complex, are employed to carry out the methods, but these represent a considerable cost factor for industrial manufacture. The solvent mixtures would have to be separated by elaborate, energy-intensive distillation methods, so that it is frequently impossible to carry out fractionation thereof economically. However, recycling of the solvents used is an indispensable precondition of industrial large-scale manufacture.

The described reactions are typically carried out with precursor concentrations of about 2 to 5%. This dilute method is uneconomic in industrial terms because such a low space-time yield would lead to higher preparation costs when carrying out manufacturing processes.

The C,O linkages described by Buchwald et al. typically start from aryl iodide compounds in order to be able to achieve conversions of 60 to 80% in the coupling reaction, but use of the more reasonably priced bromides and chlorides is to be preferred from the economic viewpoint.

In order to be able to employ fine chemicals for applications in the pharmaceuticals sector, they must often satisfy strict criteria in relation to the chemical purity and the isomer content. An efficient catalyst system that makes it possible for the aryl halides employed to be converted completely into the corresponding C,O-coupling products is necessary in order to achieve the same, since removal of precursor residues from the coupling product is often possible poorly or only with great effort. The C,O-linkage method described by Buchwald affords yields of typically only 60-80%, making it very difficult and in some cases even impossible to prepare products of high purity and free of precursors by this method.

### SUMMARY OF ADVANTAGEOUS EMBODIMENTS OF THE INVENTION

For the states reasons, it would therefore be very desirable to have a method which by employing small amounts of ligands which are not toxic or environmentally hazardous makes copper-catalyzed C,O coupling possible with a conversion which is as quantitative as possible, the intention being that the ligands be easy to remove from the organic coupling product during the workup. It would also in this connection be desirable in particular to have available a distinctly more active copper-ligand catalyst system than the systems described to date, to make it possible to reduce the reaction times from the currently typical 36-42 h and to improve markedly the space-time yields by carrying out the reaction with precursor concentrations of >10%. It was further intended that such a method provide copper-free products through the ligands to be employed being soluble in water and thus the metal entering the aqueous phase after aqueous workup.

### DETAILED DESCRIPTION OF ADVANTAGEOUS EMBODIMENTS OF THE INVENTION

The present invention achieves all these objects and provides an attractive method for coupling alcoholates for preparing compounds of the formula (III) by reacting compounds of the formula (II) with a) an alchoholate or b) an alcohol R1-OH and a base

In the presence of a Cu-containing catalyst and of a ligand, where the substituents R1 to R6, X₁ to X₅ and Hal have the following meaning:

R1 is a substituent from the group: methyl, primary, secondary or tertiary, cyclic or acyclic C₁ to C₁₂ alkyl radical, substituted cyclic or acyclic C₁ to C₁₂ alkyl radical or a phenyl, substituted phenyl, heteroaryl or substituted heteroaryl radical;

R2-5 are substituents from the group: hydrogen, methyl, primary, secondary or tertiary, cyclic or acyclic alkyl radical in which one or more hydrogen atoms are optionally replaced by fluorine or chlorine, e.g., CF₃, substituted cyclic or acyclic alkyl radicals, alkoxy, dialkylamino, alkylamino, arylamino, diarylamino, phenyl, substituted phenyl, heteroaryl, substituted heteroaryl, alkylthio, arylthio, diarylphosphino, dialkylphosphino, alkylarylphosphino, dialkyl-, arylalkyl- or diarylaminocarbonyl, monoalkyl- or monoarylaminocarbonyl, CO₂⁻, alkyl- or aryloxycarbonyl, hydroxyalkyl, alkoxyalkyl, nitro, cyano radicals, or two adjacent radicals R2-5 together form an aromatic, heteroaromatic or aliphatic

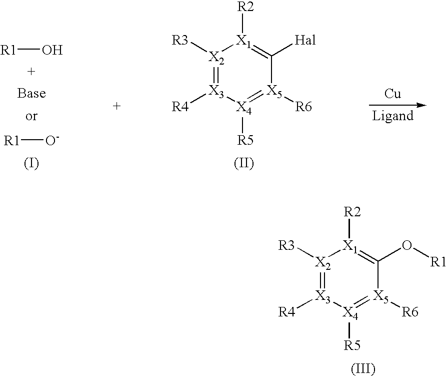

fused-on ring; Hal is chlorine, bromine, iodine, alkylsulfonate or arylsulfonate,

X₁₋₅ are independently of one another either carbon or nitrogen, or in each case two adjacent X₁R₁ linked by formal double bond are together O (furans), S (thiophenes), NRH or _{Nrl} (pyrroles).

The method is distinguished in this connection by the possibility of achieving quantitative reactions, and the reaction times typically being reduced to 8 to 12 h. Compared with conventional variants of C,O coupling, this novel method further exhibits the additional advantage that the oligoglycols and polyglycols or polyamines whatsoever and are typically not classified as dangerous goods. A further advantage of the method is that both the ligands and the copper compounds can be removed without difficulty from the respective coupling products because, on the one hand, they are often very readily soluble in water, in contrast to the organic coupling products, and, on the other hand, they often have high boiling points which prevent contamination of the coupling products during workup by distillation.

The ligands employed are acyclic and/or cyclic oligo- and polyglycols, oligo- and polyamides, or oligo- or polyamino glycols of the general formula (IV).

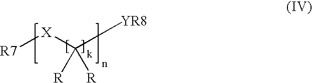

In this connection, R7-8 in formula (IV) are substituents from the group; hydrogen, methyl, primary, secondary or tertiary, cyclic or acyclic alkyl radicals in which one or more hydrogen atoms are optionally replaced by fluorine or chlorine, e.g. CF₃, substituted cyclic or acyclic alkyl groups. The groups R7 and YR8 may also together form a ring.

k is the number of [CR₈] units and n is the number of [X[CR₂]ₖ] units, where k is an integer >0 and n is an integer >1.

The heteroatoms X and Y are independently of one another oxygen or nitrogen (NH or NR₁), it being possible for them to be either simultaneously both nitrogen (NH or NR₁) and both oxygen or separately from one another oxygen or nitrogen (NH or NR₁). The radicals R in the formula (IV) are substituents of the [CR₂] units from the group: hydrogen, methyl, primary, secondary or tertiary, cyclic or acyclic alkyl radicals in which one or more hydrogen atoms are optionally replaced by fluorine or chlorine, e.g. CF₃, substituted cyclic or acyclic alkyl groups. The substituents R of adjacent [CR₂] units may also in this connection together form a double bond or be part of an aromatic ring.

The species employed as catalyst consists of copper powder or a copper compound of oxidation state 0, +1 or +2.

Alcoholates preferably employed are those in which R₁ is alkyl radicals such as methyl, ethyl, 2-propyl or tert-butyl. They alcoholate R₁-O⁻ is optionally, if commercially available, purchased or generated in a preceding step or (subsequently) formed in the reaction mixture through the presence of base during the reaction. In the latter case, the appropriate alcohols R₁—OH are reacted with commercially available bases, for example with carbonates, hydrides, hydroxides or phosphates.

Preferred haloaromatic compounds of the formula (II) which can be reacted by the method of the invention are, for example, benzenes, pyridines, pyrimidines, pyrazines, pyridazines, furans, thiophenes, pyrroles, any N-substituted pyrroles or naphthalenes. Suitable examples are 2-, 3- and 4-bromobenzotrifluoride, 2-, 3- and 4-iodobenzotrifluoride, 2-, 3-bromothiophene, 2-, 3-, 4-bromopyridine, 3,5-dimethylbromobenzene, 3,5-dimethylchlorobenzene, 3,5-dimethyliodobenzene.

Examples of suitable solvents are tetrahydrofuran, dioxane, diethyl ether, di-n-butyl ether, diisopropyl ether or alcohols such as methanol, ethanol, 2-propanol or n-butanol. Methanol, ethanol and 2-propanol are preferably employed.

The preferred reactions temperatures are between +25° C. and +200° C., and temperatures between 60 and 120° C. are particularly preferred.

The compounds employed are preferably those such as copper(I) chloride, copper(I) bromide, copper(I) iodide, copper(I) oxide, copper(II) acetylacetonate, copper(II) bromide, copper(II) iodide, particularly preferably copper(I) chloride and copper(I) bromide. It is possible in most cases to use very small amounts of copper catalyst, the amounts of copper typically employed being typically between 20 and 0.01 mol %, particularly typical amounts being between 5 and 0.05 mol %.

Typical examples of ligands according to formula diagram (IV) are compounds such as hexadecyloxypropanol, octadecyloxyethanol, hexadecyloxypropyl methyl ether, octadecyloxyethyl methyl ether, polyethylene glycol 200, polyethylene glycol 300, polyethylene glycol 600, polyethylene glycol 1000, polyethylene glycol 20,000, polyethylene glycol 35,000, polyethylene glycol dimethyl ether 250, polyethylene glycol dimethyl ether 500, polyethylene glycol dimethyl ether 2000, 1,4,10-trioxa-7,13-diazacyclopentadecane, 2-(2-aminoethoxy)ethanol, tetraethylenepentamines, and crown ethers such as 12-crown-4, 18-crown-6, benzo-15-crown-5, benzo-18-crown-6, decyl-18-crown-6, dibenzo-18-crown-6, N-phenylaza-15-crown-6, it being possible to employ one or more compounds together. It is possible in most cases to use very small amounts of ligands, the amounts of ligands typically employed being between 40 and 0.02 mol %, particularly typical amounts being between 10 and 0.1 mol %.

Aqueous workups are generally employed, with either water or aqueous mineral acids being metered in or the reaction mixture being metered into water or aqueous mineral acids. The best yields are achieved here by adjusting the pH or the product to be isolated in each case, i.e. usually a slowly acids, and in the case of heterocycles a slightly alkaline, pH. The reaction mixtures are generally filtered after the addition of water, in order to remove insoluble copper compounds. The reaction products are isolated for example by extraction and concentration of the organic phases. It is alternatively possible to remove the organic solvents from the hydrolysis mixture by distillation and to isolate the product which then precipitates by filtration.

The purities of the products from the methods of the invention are generally high, but for special applications (pharmaceuticals precursors) it is possible also to carry out a further purification step, for example by recrystallization or final distillation. The yields of reaction products are 60 to 99%, and typical yields are in particular 85 to 99%.

The method of the invention opens up a very economical method for transforming aromatic halides and sulfonates into the corresponding alkoxy- or aryloxy-substituted compounds.

The method of the invention is to be explained by the following examples without restricting the invention thereto:

### EXAMPLE 1

### Preparation of 2-methoxythiophene From 2-bromothiophene

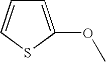

300 g of 2-bromothiophene (1.84 mol) are introduced into a mixture of 2.64 g of copper(I) bromide (CuBr, 1 mol %), 18.4 g of polyethylene glycol dimethyl ether (PEG DME) 500 (2 mol %) and 660 g of sodium methanolate solution in methanol (30% strength) (precursor concentration 30.6%) and heated to 90° C. After the conversion, determined by GC, it is >98% (total) of 8 h), the reaction mixture is added to 500 g of water. It is then filtered through Decalite, and the mixture is extracted twice with 150 g of MTBE each time. Vacuum fractionation of the combined organic phases results in 182 g of 2-methoxythiophene (1.59 mol, 86.4%), GC purity >99% a/a.

### EXAMPLE 2

### Preparation of 3-methoxythiophene From 3-bromothiophene

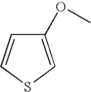

185 g of 3-bromothiophene (1.13 mol) are introduced into a mixture of 3.24 g of copper(I) bromide (CuBr, 2 mol %), 14.1 g of PEG DME 250 (5 mol %) and 407 g of sodium methanolate solution in methanol (30% strength) (precursor concentration 30.4) and heated to 90° C. After the conversion, determined by GC, it is >98% (total of 10 h), the reaction mixture is added to 300 g of water. It is subsequently filtered through decalite, and the mixture is extracted twice with 120 g of MTBE each time. Vacuum fractionation of the combined organic phases results in 117.4 g of 3-methoxylthiophene (1.03 mol, 91%), GC purity >99% a/a.

### EXAMPLE 3

### Preparation of 3,5-bis(trifluoromethyl)anisole From 1-bromo-3,5-bis(trifluoromethyl)benzene

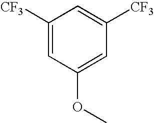

25 g of 1-bromo-3,5-bis(trifluoromethyl)benzene (85 mmol) are introduced into a mixture of 366 mg of copper(I) bromide (CuBr, 3 mol %), 1.57 g of 18-crown-6 (7 mol %) and 92.3 g of sodium methanolate solution in methanol (30% strength) (precursor concentration 21%) and heated to 105° C. After the conversion, determined by GC, it is >97% (total of 22 h), the reaction mixture is added to 175 g of water. The mixture is brought to pH 5-6 by metering in hydrochloric acid and is then filtered through decalite. The mixture if extracted twice with 150 g of dichloromethane each time. Vacuum fractionation of the combined organic phases results in 16.5 g of 3,5-bis(trifluoromethyl)anisole (68 mmol, 79.4%), GC purity >98.5% a/a.

### EXAMPLE 4

### Preparation of 2-ethoxy-3-(trifluoromethyl)pyridine From 2-bromo-3-(trifluoromethyl)pyridine

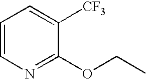

45 g of 2-bromo-3-(trifluoromethyl)pyridine (0.2 mol) are introduced into a mixture of 574 mg of copper(I) bromide (CuBr, 2 mol %), 2 g (4 mol %) of polyethylene glycol dimethyl ether 250 and 136 g of sodium ethanolate solution in ethanol (20% strength) (precursor concentration 24.5%) and heated to 100° C. After the conversion, determined by GC, it is >99% (total of 9 h), the reaction mixture is added to 125 g of water. The mixture is brought to pH 9 by metering in hydrochloric acid and then filtered through decalite. The mixture is extracted twice with 135 g of toluene each time. Vacuum fractionation of the combined organic phases results in 32.1 g of 2-ethoxy-3-(trifluoromethyl)pyridine (0.17 mmol, 84%) GC purity >99% a/a.

### EXAMPLE 5

### Preparation of 2-methoxy-5-methylpyridine From 2-bromo-5-methylpyridine

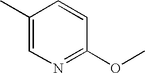

250 g of 2-bromo-5-methylpyridine (1.45 mol) are introduced into a mixture of 2.1 g of copper(I) bromide (CuBr, 1 mol %), 14.5 g (2 mol %) of polyethylene glycol dimethyl ether 500 and 457 g of sodium methanolate solution in methanol (30% strength) (precursor concentration 14.5%) and heated to 90° C. After the conversion, determined by GC, it is >98.5% (total of 17 h), the reaction mixture is added to 750 g of water. The mixture is brought to pH 9 by metering in hydrochloric acid and is then filtered through decalite. The mixture is extracted twice with 350 g of MTBE each time. Vacuum fractionation of the combined organic phases results in 162.8 g of 2-methoxy-5-methylpyridine (1.32 mol, 91%), GC purity >98% a/a.

### EXAMPLE 6

### Preparation of 2-ethoxy-3-methylthiophene From 2-bromo-3-methylthiophene

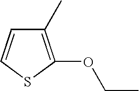

45 g of 2-bromo-3-methylthiophene (0.25 mol) are introduced into a mixture of 1.79 g of copper(I) bromide (CuBr, 5 mol %), 7.5 g (12 mol %) of polyethylene glycol dimethyl ether 250 and 255 g of sodium methanolate solution in ethanol (20% strength) (precursor concentration 14.5%) and heated to 110° C. After the conversion, determined by GC, it is >98.5% (total of 17 h), the reaction mixture is added to 600 g of water. It is then filtered through decalite, and the mixture is extracted twice with 350 g of MTBE (methyl tert-butyl ether) each time. Vacuum fractionation of the combined organic phases results in 31.3 g of 2-ethoxy-3-methylthiophene (0.22 mol, 87%), GC purity >99% a/a.

1. A method for preparing compounds of the formula (III) comprising reacting compounds of the formula (II) with a) an alcoholate or b) an alcohol R1—OH and a base 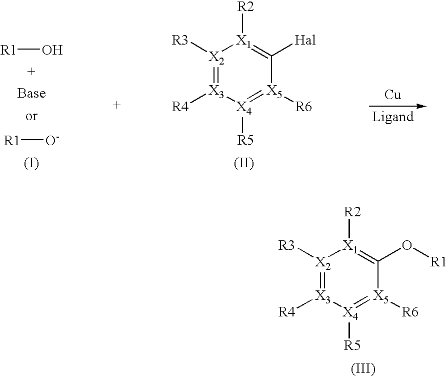
in the presence of a Cu-containing catalyst and of a ligand, wherein:
R1 is a substituent from the group: methyl, primary, secondary or tertiary, cyclic or acyclic C₁ to C₁₂ alkyl radical, substituted cyclic or acylcic C₁ to C₁₂ alkyl radical or a phenyl, substituted phenyl, heteroaryl or substituted heteroaryl radical;
R2-5 are substituted from the group: hydrogen, methyl, primary, secondary or tertiary, cyclic or acyclic alkyl radical in which one or more hydrogen atoms are optionally replaced by fluorine or chlorine, substituted cyclic or acyclic alkyl radicals, alkoxy, dialkylamino, alkylamino, arylamino, diarylamino, phenyl, substituted phenyl, heteroaryl, substituted heteroaryl, alkylthio, arylthio, diarylphosphine, dialkylphosphino, alkylarylphosphino, dialkyl-, arylalkyl- or diarylaminocarbonyl, monoalkyl- or monoarylaminocarbonyl, CO₂⁻, alkyl- or aryloxycarbonyl, hydroxyalkyl, alkoxyalkyl, nitro, cyano, radicals, or two adjacent radicals R2-5 together form an aromatic, heteroaromatic or aliphatic fused-on ring;
Hal is chlorine, bromine, iodine, alkylsulfonate or arylsulfonate, and
X₁₋₅ are independently of one another either carbon or nitrogen, or in each case two adjacent X₁R₁ linked by formal double bond are together O, S, NRH or NR₁.

2. The method as claimed in claim 1, wherein the ligands are acyclic and/or cyclic oligo- and polyglycols, oligo- and polyamides or oligo- and polyamino glycols of the general formula (IV) 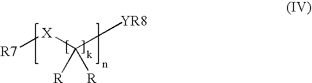
where R7-8 are substitutents from the following group: hydrogen, methyl, primary, secondary or tertiary, cyclic or acylcic alkyl radical in which optionally one or more hydrogen atoms are replaced by fluorine or chlorine, substituted cyclic or acyclic alkyl radicals or the groups R7 and YR8 together form a ring;
k is an integer >0 and n is an integer >1;
X and Y are independently of one another O, NH or NR₁,
R is a substituent from the group: hydrogen, methyl, primary, secondary, tertiary, cyclic or acyclic alkyl radical in which optionally one or more hydrogen atoms are replaced by fluorine or chlorine, or substituted cyclic or acyclic alkyl radicals or the substituents R of adjacent CR₂ units together form a double bond and are an alkenyl radical or are part of an aromatic ring.

3. The method as claimed in claim 1, wherein the ligand comprises one or more compounds of the following group: hexadecyloxypropanol, octandecyloxyethanol, hexadecyloxypropyl methyl ether, octadecyloxyethyl methyl ether, polyethylene glycol 200, polyethylene glycol 300, polyethylene glycol 600, polyethylene glycol 1000, polyethylene glycol 20,000, polyethylene glycol 35,000, polyethylene glycol dimethyl ether 250, polyethylene glycol dimethyl ether 500, polyethylene glycol dimethyl ether 2000, 1,4,10-trioxa-7,13-diazacyclopentadecane, 2-(2-aminoethoxy)ethanol, tetraethylenepentamines, or crown ethers.

4. The method as claimed in claim 1, wherein the catalyst comprises copper in oxidation state 0, +1 or +2.

5. The method as claimed in claim 4, wherein the catalyst comprises copper(I) chloride, copper(I) bromide, copper(I) iodide, copper(I) oxide, copper(II) acetylacetonate, copper(II) bromide, copper(II) iodide or copper powder.

6. The method as claimed in claim 1, wherein the catalyst comprises copper in the range from 20 to 0.01 mol %.

7. The method as claimed in claim 1, wherein the ligand is present in amounts of from 40 to 0.02 mol.

8. The method as claimed in claim 1, wherein the starting compounds of the formula (II) are benzenes, pyridines, pyrimidines, pyrazines, pyridazines, furans, thiophenes or optionally substituted pyrroles or naphthalenes.

9. The method as claimed in claim 1, wherein the method is carried out in a solvent from the group: tetrahydrofuran, dioxane, diethyl ether, di-n-butyl ether, diisopropyl ether, methanol, ethanol, 2-propanol or n-butanol.

10. The method as claimed in claim 1, wherein the method is carried out at a temperature in the range from +25° C. to +200° C.
